(19) United States
(12) Patent Application Publication
(10) Pub. No.: US 2012/0294818 A1
(43) Pub. Date: Nov. 22, 2012

(54) Antiperspirant Compositions

(75) Inventors: Hani Fares, Somerset, NJ (US); Christine M. Barrett, Oakland, NJ (US); Anthony Luschen, Wayne, NJ (US); Rita Marie Guerrero, Morris Plains, NJ (US); Tracey Ross, Hewitt, NJ (US)

(73) Assignee: ISP INVESTMENTS INC., Wilmington, DE (US)

(21) Appl. No.: 13/254,932

(22) PCT Filed: Mar. 11, 2010
(86) PCT No.: PCT/US2010/026940

§ 371 Date: Apr. 17, 2012

Related U.S. Application Data
(60) Provisional application No. 61/159,284, filed on Mar. 11, 2009.
(60) Provisional application No. 61/173,399, filed on Apr. 28, 2009.

(51) Int. Cl. A61K 8/81; A61Q 15/00

(52) U.S. Cl. 424/66; 424/65; 424/68; 424/67

(57) ABSTRACT

Compositions containing a relatively high concentration of electrolytes and a thickening agent, wherein the thickening agent comprises a strongly swellable, lightly to moderately crosslinked polyvinyl pyrrolidone. The polyvinyl pyrrolidone thickening agent is characterized by an aqueous gel volume of about 15 to 150 ml/g and a Brookfield viscosity of at least 10,000 cps for a 5% aqueous solution at 25° C.

### CROSS REFERENCE TO RELATED APPLICATIONS

This application claims the benefit of U.S. Provisional App. Ser. No. 61/159,284 filed Mar. 11, 2009 and U.S. Provisional App. Ser. No. 61/173,399 filed Apr. 28, 2009, the contents of which are hereby incorporated by reference.

### FIELD

The present application relates to antiperspirant and/or deodorant compositions, and, more particularly, to compositions containing a strongly swellable, lightly to moderately crosslinked polyvinyl pyrrolidone.

### BACKGROUND

Cosmetic and personal care compositions containing relatively high concentrations of electrolytes are difficult to formulate as true gels or as oil-in-water emulsions which exhibit cosmetically acceptable aesthetics and good stability. Compositions containing high concentrations of electrolytes can be difficult to thicken and tend to be unstable over time. Most polymers will salt out of a composition containing a high concentration of electrolytes which results in a loss of viscosity over time. Antiperspirant compositions, including antiperspirant gels and oil-in-water emulsions, typically contain high concentrations of electrolytes, and, therefore, these compositions can exhibit undesirable loss of viscosity over time and are difficult to thicken with typical polymeric thickening agents.

Antiperspirant compositions are well known in the art. See, e.g., U.S. Pat. Nos. 4,985,238; 5,302,381; 5,376,362; 5,417,963; 5,482,702; and 5,486,355, the disclosures of which are hereby incorporated by reference. The active antiperspirant ingredient in such compositions usually is an inorganic compound, e.g., an aluminum, zirconium, or zinc salt such as an aluminum zirconium tetrachlorohydrate complex with glycine. Currently marketed gels are actually transparent water-in-oil or water-in-silicone emulsions wherein the continuous phase and the dispersed phase are formulated to have approximately the same refractive index. The preparation of such emulsions is relatively expensive and time consuming Cellulosic derivatives have been incorporated into some antiperspirant compositions in an effort to enhance stability, but these compositions still exhibit less than desirable stability and fail to provide the desirable aesthetic properties.

### SUMMARY

The present application relates to compositions containing a relatively high concentration of electrolytes and a thickening agent, wherein the thickening agent comprises a strongly swellable, lightly to moderately crosslinked polyvinyl pyrrolidone. The polyvinyl pyrrolidone thickening agent is characterized by an aqueous gel volume of about 15 to 150 ml/g and a Brookfield viscosity of at least 10,000 cps for a 5% aqueous solution at 25° C.

In accordance with one embodiment of the present invention, the composition is an antiperspirant and/or deodorant composition containing at least one of an active antiperspirant ingredient and a deodorant active. The antiperspirant/deodorant composition as described herein may take the form of a stick, lotion, cream, roll-on, solution or aerosol. In accordance with particular aspects of the present invention, the active antiperspirant ingredient may be an activated aluminum chlorohydrate, degraded activated aluminum chlorohydrate, aluminum chlorohydrate or aluminum zirconium tetrachlorohydrex glycine.

In accordance with another aspect, a method of producing a stable antiperspirant composition is disclosed. The method comprises adding a thickening agent comprising a strongly swellable, lightly to moderately crosslinked polyvinyl pyrrolidone to a base composition containing an antiperspirant active ingredient.

### DETAILED DESCRIPTION

The present application relates to compositions containing a high concentration of electrolytes and a thickening agent, wherein the thickening agent comprises a strongly swellable, lightly to moderately crosslinked polyvinyl pyrrolidone. More particularly, the present application discloses antiperspirant compositions containing a relatively high concentration of electrolytes and the lightly to moderately crosslinked polyvinyl pyrrolidone thickening agent.

The compositions in accordance with the present invention contain relatively high concentrations of electrolytes. Electrolytes useful in the compositions disclosed herein are not particularly limited and include, without limitation, materials such as antiperspirant actives, deodorant actives and thioglycolates. In accordance with certain aspects of the present invention, the electrolytes are present at a concentration of about 5% to about 35%, more particularly of about 7% to about 30%, still more particularly at about 10% to about 25%, and in accordance with certain embodiments from about 12% to about 20% based on the weight of the composition.

Various active antiperspirant ingredients that can be utilized according to the present invention include conventional antiperspirant metal salts and complexes of metal salts. In one aspect of the invention the metal salts and metal salt complexes utilized as the active antiperspirant ingredients are acidic and are based on aluminum and zirconium and combinations thereof. These salts include but are not limited to aluminum halides, aluminum hydroxyhalides, aluminum sulfate, zirconium (zirconyl) oxyhalides, zirconium (zirconyl)hydroxyhalides, and mixtures or complexes thereof. Complexes of aluminum and zirconium salts include aluminum and zirconium salt complexes with amino acids, such as, for example, glycine or complexes with a glycol, such as, for example, propylene glycol (PG) or polyethylene glycol (PEG). Exemplary active antiperspirant ingredients include, but are not limited to, aluminum chloride, aluminum chlorohydrate, activated aluminum chlorohydrate, degraded activated aluminum chlorohydrate, aluminum dichlorohydrate, aluminum sesquichlorohydrate, zirconyl hydroxychloride, aluminum chlorohydrex PEG (aluminum chlorohydrex polyethylene glycol), aluminum chlorohydrex PG (aluminum chlorohydrex propylene glycol), aluminum dichlorohydrex PEG (aluminum dichlorohydrex polyethylene glycol), aluminum dichlorohydrex PG (aluminum dichlorohydrex propylene glycol), aluminum sesquichlorohydrex PEG (aluminum sesquichlorohydrex polyethylene glycol), aluminum sesquichlorohydrex PG (aluminum sesquichlorohydrex propylene glycol), aluminum zirconium trichlorohyrate, aluminum zirconium tetrachlorohyrate, aluminum zirconium pentachlorohyrate, aluminum zirconium octachlorohyrate, aluminum zirconium chlorohydrex GLY (aluminum zirconium chlorohydrex glycine), aluminum zirconium trichlorohydrex GLY (aluminum zirconium trichlorohydrex glycine), aluminum zirconium tetrachlorohyrex GLY (aluminum zirconium tetrachlorohyrex glycine), aluminum zirconium pentachlorohyrex GLY (aluminum zirconium pentachlorohyrex glycine), and aluminum zirconium octachlorohyrex GLY (aluminum zirconium octachlorohyrex glycine). Other active antiperspirant ingredients include ferric chloride and zirconium powder. Mixtures of any of the foregoing active antiperspirant ingredients are also suitable for use in the present invention.

The amount of the active antiperspirant ingredients incorporated into the compositions of the present invention is an amount that is sufficient to reduce the flow of perspiration from the location to which the antiperspirant product is applied, for example to the axillary area of the human body.

The deodorant active used in accordance with certain embodiments can be any deodorant active known in the art such as alcohols, antimicrobial actives such as polyhexamethylene biguanides, non-microbiocidal deodorant actives such as triethylcitrate, bactericides and bacteriostats. Specific examples of deodorant actives include, but are not limited to, 2,4,4′-trichloro-2′-hydroxy diphenyl ether (Triclosan), octoxyglycerin (SENSIVA™ SC 50), benzethonium chloride, triethylcitrate, 2-amino-2-methyl-1-propanol (AMP), cetyl-trimethylammomium bromide, and cetyl pyridinium chloride. One or more active can be combined to produce antiperspirant/deodorant compositions as described herein. Deodorant actives may be used in the same amounts as the antiperspirant actives.

Generally, the level of active antiperspirant ingredients utilized in the compositions of the present invention ranges from about 0.5 wt. % to about 35 wt. % based on the total weight of the composition. In another aspect of the invention, the amount of active antiperspirant ingredient in the composition can range from about 5 wt. % to about 25 wt. %, in a further aspect from about 5 wt. % to about 20 wt. %, and in a still further aspect from about 10 wt. % to about 15 wt. %, based on the total weight of the composition. The foregoing weight percentages are calculated on an anhydrous metal salt basis.

The compositions described herein also include a thickening agent comprising a strongly swellable, lightly to moderately crosslinked polyvinyl pyrrolidone such as those described in commonly assigned U.S. Pat. Nos. 5,139,770 and 5,312,619, which are incorporated herein by reference. These thickening agents typically contain from about 0.25% to about 1% by weight of a crosslinking agent selected from the group consisting of divinyl ethers and diallyl ethers of terminal diols containing from about 2 to about 12 carbon atoms, divinyl ethers and diallyl ethers of polyethylene glycols containing from about 2 to about 600 units, dienes having from about 6 to about 20 carbon atoms, divinyl benzene, vinyl and allyl ethers of pentaerythritol, and the like.

The term “strongly swellable, lightly to moderately crosslinked PVP”, unless otherwise noted, specifically refers to polymer essentially consisting of lightly- to moderately-crosslinked poly(N-vinyl-2-pyrrolidone) having at least one of the following characteristics: (1) an aqueous swelling parameter defined by its gel volume from about 15 mL/g to about 300 mL/g, more particularly from about 15 mL/g to about 250 mL/g, and in other cases from about 15 mL/g to about 150 mL/g, or (2) a Brookfield viscosity of 5% crosslinked PVP in a liquid carrier comprising water at 25° C. of at least 2,000 cP, more particularly of at least about 5,000 cP, and in certain cases of at least about 10,000 cP. Disclosure for these parameter ranges is provided in U.S. Pat. No. 5,073,614 (incorporated herein by reference) and in Shih, J. S., et al. (1995). Synthesis methods for the crosslinked PVP are disclosed in a number of references, including U.S. Pat. Nos. 5,073,614; 5,654,385; and 6,177,068, the contents of which are hereby incorporated by reference. It is appreciated by a polymer scientist skilled in the art that the method of synthesis is immaterial, inasmuch as the produced polymer achieves at least one of the above defined parameters.

For example, U.S. patent '614 discloses different crosslinkers and crosslinker amounts that yield crosslinked PVP suitable for the present invention. The effect of crosslinker amount on swell volume and viscosity is graphically presented in Shih, J. S., et al. (1995). Thus, the crosslinked PVP may be produced by the precipitation polymerization method of the '614 patent, by the hydrogel method described in the '385 patent, or by the non-aqueous, heterogeneous polymerization method of the '068 patent. Certainly, other techniques are contemplated to synthesize this polymer, provided the product meets the aqueous swelling parameter and Brookfield viscosity requirements.

Final product viscosities may slightly vary for compositions containing crosslinked PVP made by these different methods. Nonetheless, these variations are within the scope of the invention, as the crosslinked PVPs thicken low pH compositions.

Unless otherwise specified, “strongly swellable, lightly to moderately crosslinked PVP” does not refer to swellable but water-insoluble crosslinked PVP, such as the type sold into commercial trade under the trade name Polyclar by International Specialty Products, which differs from the above described crosslinked PVP.

Commercially available examples of strongly swellable, lightly to moderately crosslinked PVP include, but are not limited to, ACP-1120, ACP-1179, and ACP-1180, available from International Specialty Products (Wayne, N.J.).

As recognized by one of ordinary skill in the art, the amount of thickening agent can vary depending upon the consistency and the desired thickness of the composition. In accordance with certain aspects of the present invention, the thickening agent may be used in amounts from about 0.5 to about 10%, more particularly from about 1% to about 7%, and in accordance with certain aspects of the invention from about 2% to about 5% by weight of the composition.

Compositions in accordance with certain aspects of the present invention may be formulated with various other excipients as known to those of ordinary skill in the art to improve performance.

In accordance with some aspects of the present application, compositions are disclosed that exhibit viscosity stability even though the compositions contain a relatively high concentration of electrolytes. Compositions in accordance with some aspects exhibit only slight drops in viscosity as the electrolyte concentration of the composition is increased. For example, compositions containing the thickening agent described herein may have an electrolyte such as aluminum chlorohydrate (ACH) added in amounts from about 1% to about 5% with only slight changes in viscosity of the composition. In accordance with a particular composition, the viscosity of the control composition without any ACH was about 26,000 cps and the viscosity was maintained within about 5000 cps of that value for compositions containing 1%, 2%, 3%, 4% and 5% ACH.

In accordance with certain embodiments, the loss of viscosity for compositions containing up to about 5% electrolytes or even more may be less than about 35%, more particularly less than about 25% and in certain cases less than about 20%, 15% or even 10% compared to a control composition without any electrolytes.

Compositions produced in accordance with certain aspects may be tested for stability as determined by the stability test set forth in the examples below. In accordance with particularly useful compositions, the compositions may be considered stable when the viscosity of the composition under different conditions is maintained at a viscosity at least as high as the initial viscosity of the composition. In accordance with certain aspects, stability can be determined by measuring the viscosity over a period of three months when stored at 45° C. Of course, stability for any particular application can depend on the end use such that other compositions may still be considered stable even though the viscosity decreases to some extent with time or under certain conditions. Stability can also be determined under other conditions such as those set forth below. Stability of the composition can also be based on freeze thaw cycling as described below and can be based on the stability of the pH of the composition under different conditions.

Having generally described the invention, reference is now made to the accompanying examples which set forth particular embodiments, but which are not to be construed as limiting to the scope of the invention as more broadly set forth above and in the appended claims.

### Example 1

Formula Name: AZG 1120 AP Emulsion
NB#11721-135-1; Batch size 400

| | |
|---|---|
| Ingredients | % W/W |

Phase A

| | | |
|---|---|---|
| Water | 72.75% | 291.00 |
| AZG (Aluminum Zirconium | 15.00% | 60.00 |
| Tetrachlorohydrex Glycine) | | |
| ACP 1120 (thickening agent) | 2.00% | 8.00 |

Phase B

| | | |
|---|---|---|
| Si-Tec CM040 (cyclopentasiloxane) | 4.00% | 16.00 |
| Ceraphyl SLK (isodecyl | 2.00% | 8.00 |
| Ncopentanoate) | | |
| Ceraphyl 31 (Lauryl lactate) | 2.00% | 8.00 |
| Cerasynt 945 (glycerol stearate (and) | 1.25% | 5.00 |
| Laureth-23) | | |
| PEC-20 Stearate | 1.00% | 4.00 |
| Total | 100.00% | 400.00 |

Procedure:

1. Add water to main vessel.
2. Add AZG under homogeneous mixing until uniform.
3. Add ACP 1120 and homogeneous mix until uniform while heating to 55° C.
4. Separately, heat Phase B to 55° C.
5. Add phase B to Phase A and continue to homogeneous mix, cooling to RT.
Note: This sample separated at 45° C. and testing was halted.

### Example 2

Formula Name: ACP 1120 AP Gel
NB#11721-125-1; Batch size 1,000

| | |
|---|---|
| Ingredients | % W/W |

| | | |
|---|---|---|
| Water | 64.0% | 644 |
| EtOH | 9.20% | 92 |
| ACH (aluminum chlorohydrate) | 13.80% | 138 |
| ACP 1120 | 4.60% | 46 |
| Si-Tec CM040 (cyclopentasiloxane) | 4.00% | 40 |
| Ceraphyl SLK (isodecyl | 2.00% | 20 |
| Neopentanoate) | | |
| Ceraphyl 31 (lauryl lactate) | 2.00% | 20 |
| Total | 100.00% | 1000.00 |

Procedure:

1. Add water and alcohol to main vessel.
2. Add ACH under homogeneous mixing until uniform.
3. Add ACP 1120 and homogeneous mix until uniform.
4. Add remaining ingredients, mixing well between additions.

Stability Testing for Example 2 (11721-125-1)

The initial viscosity and pH of the composition were measured and then samples were stored under three temperature conditions (5° C., room temperature, and 45° C.). Samples from each condition were pulled after various time periods for retesting. All samples were equilibrated overnight to room temperature before taking measurements. The sample was initially characterized as a semi-transparent gel. The appearance remained relatively unchanged except for the samples stored at 45° C. which appeared slightly yellow after 4 weeks.

Initial viscosity—30,000 cps Initial pH—4.19

| | RT | | 5° C. | | 45° C. | |
|---|---|---|---|---|---|---|
| Pulls | viscosity | pH | viscosity | pH | viscosity | pH |
| 2 weeks | 52,000 | 4.28 | 47,000 | 4.4 | 55,500 | 4.43 |
| 4 weeks | 57,500 | 4.57 | 53,000 | 4.6 | 59,500 | 4.56 |
| 2 months | 50,000 | 4.04 | 60,500 | 4.21 | 59,500 | 4.22 |
| 3 months | 55,000 | 4.47 | 56,000 | 4.52 | 59,000 | 4.46 |

Brookfield Viscosity TC@10 RPM (cps)

The samples also exhibited stability under freeze thaw cycling. One freeze thaw cycle is 24 hrs at −10° C. and 24 hrs at room temperature (RT).

| | Freeze Thaw Cycles | | | | | |
|---|---|---|---|---|---|---|
| | initial | Cycle 1 | Cycle 2 | Cycle 3 | Cycle 4 | Cycle 5 |
| Viscosity TC@ | 30,000 | 42,500 | 47,500 | 47,000 | 48,500 | 56,000 |
| 10 RPM (CPS) | | | | | | |
| pH | 4.19 | 4.19 | 4.23 | 4.32 | 4.72 | 4.26 |
| Appearance | Semi- | Semi- | Semi- | Semi- | Semi- | Semi- |
| | Transparent | Transparent | Transparent | Transparent | Transparent | Transparent |
| | Gel | Gel | Gel | Gel | Gel | Gel |

### Example 3

Formula Name: ACP 1120 AP Gel
NB#11721-130-1; Batch size 200

| | |
|---|---|
| Ingredients | % W/W |

| | | |
|---|---|---|
| Water | 72.0% | 144 |
| EtOH | 10.00% | 20 |
| Gantrez ® MS955 (mixed sodium and | 4.00% | 8 |
| calcium salt of poly(methyl vinyl | | |
| ether/maleic anhydride)) | | |
| Zano 10 (zinc oxide) | 1.00% | 2 |
| ACP 1120 | 5.00% | 10 |
| Si-Tec CM040 (cyclopentasiloxane) | 4.00% | 8 |
| Ceraphyl SLK (isodecyl | 2.00% | 4 |
| Ncopentanoate) | | |
| Ceraphyl 31 (lauryl lactate) | 2.00% | 4 |
| Total | 100.00% | 200.00 |

Procedure:

1. Add water and alcohol to main vessel.
2. Add Gantrez and Zano under homogeneous mixing until uniform.
3. Add ACP 1120 and homogeneous mix until uniform.
4. Add remaining ingredients, mixing well between additions.
Note: This sample was unacceptable aesthetically and was not put on stability testing.

### Example 4

Formula Name: ACP 1120 AP Gel
NB#11721-130-2; Batch size 400

| | |
|---|---|
| Ingredients | % W/W |

| | | |
|---|---|---|
| Water | 74.0% | 296 |
| EtOH | 10.00% | 40 |
| Gantrez ® MS955 (mixed sodium and | 4.00% | 16 |
| calcium salt of poly(methyl vinyl | | |
| ether/maleic anhydride)) | | |
| Zano 10 (zinc oxide) | 1.00% | 4 |
| ACP 1120 | 3.00% | 12 |
| Si-Tec CM040 (cyclopentasiloxane) | 4.00% | 16 |
| Ceraphyl SLK (isodecyl | 2.00% | 8 |
| Neopentanoate) | | |
| Ceraphyl 31 (lauryl lactate) | 2.00% | 8 |
| Total | 100.00% | 400.00 |

Procedure:

1. Add water and alcohol to main vessel.
2. Add Gantrez and Zano under homogeneous mixing until uniform.
3. Add ACP 1120 and homogeneous mix until uniform.
4. Add remaining ingredients, mixing well between additions.
Note: This sample was unacceptable aesthetically and were not put on stability testing.

### Example 5

Formula Name: Aerosol AP with ACP 1120
NB#11721-134-1; Batch size 1400

| | |
|---|---|
| Ingredients | % W/W |

Phase A

| | | |
|---|---|---|
| Ceraphyl 41 | 15.00% | 210 |
| Sitec CM 040 | 7.00% | 98 |
| ACP 1120 (thickening agent) | 2.00% | 28 |

Phase B

| | | |
|---|---|---|
| Gantrez ® MS955 | 4.00% | 56 |
| Zano 10 Plus | 1.00% | 14 |
| Total | 29.00% | 406.00 |

Procedure:

1. Add Ceraphyl 41 and 040 to main vessel.
2. Add ACP 1120 under homogeneous mixing until uniform.
3. Add Phase B, mixing between each until uniform.
4. The rest of the formulation is solvent (gas) of the aerosol.
Note: This sample was unacceptable as an aerosol and was not put on stability testing.

### Example 6

Formula Name: ACP 1120 AP Gel
NB#11721-114-1; Batch size 600

| | |
|---|---|
| Ingredients | % W/W |

| | | |
|---|---|---|
| Water | 70.00% | 420 |
| EtOH | 10.00% | 60 |
| ACH | 15.00% | 90 |
| ACP 1120 | 5.00% | 30 |
| Total | 100.00% | 600.00 |

Procedure:

1. Add water and alcohol to main vessel.
2. Add ACH under homogeneous mixing until uniform.
3. Add ACP 1120 and homogeneous mix until uniform.

Stability Testing for Example 2 (11721-114-1)

The initial viscosity and pH of the composition were measured and then samples were stored under three temperature conditions (5° C., room temperature, and 45° C.). Samples from each condition were pulled after various time periods for retesting. All samples were equilibrated overnight to room temperature before taking measurements. The sample was initially characterized as a semi-transparent white gel. The appearance remained relatively unchanged except for the sample stored at 45° C. which appeared very slightly yellow after 3 months.

Initial viscosity—40,000 cps Initial pH—4.34

| | RT | | 5° C. | | 45° C. | |
|---|---|---|---|---|---|---|
| Pulls | viscosity | pH | viscosity | pH | viscosity | pH |
| 2 weeks | 59,000 | 4.51 | 54,000 | 4.51 | 66,000 | 4.49 |
| 4 weeks | 60,500 | 4.51 | 53,500 | 4.52 | 71,000 | 4.57 |
| 2 months | 66,000 | 4.11 | 61,000 | 4.27 | 64,000 | 4.28 |
| 3 months | 65,000 | 4.16 | 53,000 | 4.50 | 64,000 | 4.48 |

Brookfield Viscosity TC@10 RPM (cps)

The samples also exhibited stability under freeze thaw cycling. One freeze thaw cycle is 24 hrs at −10° C. and 24 hrs at room temperature (RT).

| | Freeze Thaw Cycles | | | | | |
|---|---|---|---|---|---|---|
| | initial | Cycle 1 | Cycle 2 | Cycle 3 | Cycle 4 | Cycle 5 |
| Viscosity TC@ | 40,000 | 49,500 | 55,000 | 54,000 | 56,500 | 52,500 |
| 10 RPM (CPS) | | | | | | |
| pH | 4.34 | 4.30 | 4.30 | 4.01 | 3.97 | 4.40 |
| Appearance | Semi- | No | No | No | No | No |
| | Transparent | Change | Change | Change | Change | Change |
| | White Gel | | | | | |

### In-Vitro Antiperspirant Efficacy Method

### Materials

1 oz. glass SS jar.
Lid with polyethylene liner with 1″ dia. circular cutout in both, centered.
Large and small polyester mesh circles (7.5 cm dia. and 4.0 cm dia.) 54 nm pore size.
2″ wide transparent tape.
Aluminum weight dish (2.5″ dia.)
Water soluble starch.
Solution of 5.0% I₂, 3.0% KI in EtOH.
Artificial Perspiration.
Hunter LAB Ultrascan Pro Colorimeter.
Thermotron Industries humidity and temperature control chamber.

### Method

1. Add 10 g Artificial Perspiration to glass jar.
2. Antiperspirant formula is applied to large polyester mesh circle. This is done in triplicate.
3. Treated mesh circle is conditioned for 1 hour @ 38° C. and 35% RH horizontally on top of aluminum weigh dish.
4. A small polyester mesh circle is coated with Iodine solution and dried.
5. The product treated mesh circle is placed over the top of the 1 oz. SS jar.
6. The Iodine treated mesh circle is placed in the lined lid.
7. The lid is then secured to the jar.
8. A transparent tape strip is cut to a length of approx. 4-5″.
9. An open ended tube of 1″ dia. is centered on the tape and starch is deposited in the tube.
10. Shake the tube/tape to evenly deposit the starch on the tape and, inverting the tube/tape, tap out the excess starch.
11. Remove the tube from the tape and place the tape, starch side down, on top of the jar with lid so that the 1″ starch circle is aligned with the cutout on the lid and seal as well as possible around the edges.
12. Assembled test jar is then placed in a 32° C. oven for 2 hours.
13. The tape strip is removed from the jar and placed on white pasteboard. The color of the starch circle will vary from white (effective) to dark purple (ineffective).
14. LAB values are read on colorimeter.

Sample 11721-125-1 when tested in accordance with the above procedure resulted in a ΔE* value of 20.45 as compared to a blank sample that resulted in a ΔE* value of 50.26. The lower ΔE* indicates improved performance of the formulation. In accordance with certain embodiments, compositions produced in accordance with the present disclosure provide ΔE* values of less than about 40, more particularly less than about 30 and in certain cases less than about 25 when tested in accordance with the above procedure.

Having described the invention in detail and by reference to disclosed embodiments thereof, it will be apparent that modifications and variations are possible without departing from the scope of the invention as defined in the following claims:

What is claimed is:

1. A composition comprising:
at least one active ingredient selected from the group consisting of an antiperspirant active and a deodorant active; and
a thickening agent, wherein the thickening agent comprises a strongly swellable, lightly to moderately crosslinked polyvinyl pyrrolidone.

2. A composition according to claim 1 wherein the concentration of the active ingredient is 5% or more.

3. A composition according to claim 1 wherein the thickening agent is characterized by an aqueous gel volume of about 15 to 150 ml/g of polymer and a Brookfield viscosity of at least 10,000 cps for a 5% aqueous solution at 25° C.

4. A composition according to claim 1 wherein the active ingredient is an active antiperspirant active.

5. A composition according to claim 4 wherein said active antiperspirant ingredient is an aluminum, zirconium, or zinc salt, or mixtures thereof.

6. A composition according to claim 5 wherein said antiperspirant ingredient is selected from the group consisting of activated aluminum chlorohydrate, degraded activated aluminum chlorohydrate, aluminum chlorohydrate, aluminum zirconium tetrachlorohydrex glycine, and mixtures thereof.

7. A composition according to claim 1 wherein the composition is in the form of a gel or oil-in-water emulsion.

8. A composition according to claim 7 wherein the composition maintains a viscosity at least as high as the initial viscosity of the composition over a period of three months when stored at 45° C.

9. A composition comprising a relatively high concentration of electrolytes and a thickening agent, wherein the thickening agent comprises a strongly swellable, lightly to moderately crosslinked polyvinyl pyrrolidone.

10. The composition of claim 9 wherein the concentration of the electrolytes is 5% or more.

11. The composition of claim 9 wherein the thickening agent is characterized by an aqueous gel volume of about 15 to 150 ml/g of polymer and a Brookfield viscosity of at least 10,000 cps for a 5% aqueous solution at 25° C.

12. The composition of claim 11 wherein the electrolytes comprise antiperspirant actives.

13. The composition of claim 12 wherein the concentration of the antiperspirant actives is 5% or more.

14. A method of producing a stable antiperspirant composition comprising:
adding a thickening agent comprising a strongly swellable, lightly to moderately crosslinked polyvinyl pyrrolidone to a base composition containing an antiperspirant active ingredient.

15. The method of claim 14 wherein the thickening agent is characterized by an aqueous gel volume of about 15 to 150 ml/g of polymer and a Brookfield viscosity of at least 10,000 cps for a 5% aqueous solution at 25° C.
